(19) United States
(12) Patent Application Publication
(10) Pub. No.: US 2022/0074917 A1
(43) Pub. Date: Mar. 10, 2022

(54) SPECTROSCOPIC METHOD AND APPARATUS FOR DETECTION OF VIRUSES AND OTHER BIOLOGICAL PATHOGENS

(71) Applicant: Brewmetrix LLC, Incline Village, NV (US)

(72) Inventor: Sydney Sukuta, Patterson, CA (US)

(73) Assignee: Brewmetrix LLC, Incline Village, NV (US)

(21) Appl. No.: 17/468,627

(22) Filed: Sep. 7, 2021

Related U.S. Application Data
(60) Provisional application No. 63/075,804, filed on Sep. 8, 2020.

(51) Int. Cl. G01N 33/487; G01N 21/31; G01N 33/497

(52) U.S. Cl. CPC G01N 33/487; G01N 2033/4975; G01N 33/497; G01N 21/31

(57) ABSTRACT

The presence of a virus, such as a coronavirus, or other pathogen can be determined through spectroscopic methods. In one example, the method includes obtaining a biological sample from a subject and obtaining spectrographic data from the sample in an onsite portable spectroscopic unit. The spectroscopic data may be transmitted to a remote lab to determine the presence of the virus or other pathogen within the sample, providing a simple to understand result that does not require significant interpretation by a medical practitioner Contrary to existing test methods such as PCR, this method has no supply chain requirements other than distilled water and results are out in real-time at a highly subdued cost.

### CROSS REFERENCE TO RELATED APPLICATIONS

This application claims priority to U.S. Provisional Patent Application No. 63/075,804 filed 8 Sep. 2020, the contents of which are herein incorporated by reference.

### FIELD OF THE INVENTION

The present invention relates to analytical systems and in particular to systems and methods for performing analysis of biological samples to determine the presence of a virus or similar biological pathogen.

### BACKGROUND

When it comes to analyzing biological samples to determine the presence of viruses and similar pathogens, a subject, either human or animal, is required to provide a biological sample that is sent to a lab for analysis. There can therefore be issues with the time taken to analyze and provide results. For example, an infectious subject may spread the pathogen while awaiting results. Thus, it would be preferable if results could be generated and provided in near real-time and in particular at the point of care.

Globally, large-scale testing is a major limiting factor in the fight to slow down the spread of COVID-19 and other viruses. Shortage of testing supplies and logistical challenges can hinder the ability to do adequate testing. In addition, large-scale testing at centralized testing locations can create an infection risk. A widely used diagnostic method is the RealTime Reverse Transcriptase (RT)-PCR assay. Other rapid nucleic acid-based tests are available, such as Abbott's ID NOW COVID-19 test. In addition to nucleic acid detection, several antigen and antibody detection assays with varying sensitivities and specificities are available. For example, the selection of SASR-CoV-2 specific IgG and IgM antibodies in human serum has been developed by many manufacturers. However, most of these existing diagnostic methods depend on laboratory infrastructure, expensive reagents (primers, probes, antibodies etc), laboratory supplies, and trained personnel, thereby limiting the scalability of the testing. For many of these tests, there is time delay in getting test results out, as the samples must be sent to an external laboratory. In some cases, the turnaround times can be of the order of 2-3 days.

What is required is an improved system and method for providing pathogen testing.

### SUMMARY OF ONE EMBODIMENT OF THE INVENTION

### Advantages of One or More Embodiments of the Present Invention

The various embodiments of the present invention may, but do not necessarily, achieve one or more of the following advantages:

the ability to detect viruses and other biological pathogens in a subject;

the ability to detect viruses in a subject with minimal equipment;

the ability to detect viruses in a subject at a point of care;

minimize prediction errors, particularly when the response variable exhibits are non-linear;

provide flexibility for the user to retrieve results from any location from which the internet can be accessed;

provide results in a user friendly manner that non-experts can easily understand;

provide a sampling method effective for lung-borne pathogens;

provide users with the option to access results at various stages of the analytical process; and

the ability to allow users the flexibility to conduct examinations and to analyze results from a location remote from the substance in question.

These and other advantages may be realized by reference to the remaining portions of the specification, claims, and abstract.

### Brief Description of One Embodiment of the Present Invention

In one aspect, the invention provides a method of determining the presence of one or more biological pathogens in a subject. The method may comprise obtaining a biological sample from the subject, obtaining a spectrograph from the biological sample, determining from the spectrograph, the presence of the biological pathogens in the subject.

In one aspect, the invention provides a system for determining the presence of a virus in a subject. The system may include spectrographic apparatus for generating spectrographic data from a biological sample from the subject, and one or more processing systems for processing the spectrographic data to determine a presence of a biological pathogen in the subject.

In one aspect, the invention provides a sensor unit. The sensor unit comprises a sample holder for receiving a biological sample, a light source configured to direct light into the biological sample, and a spectrometer for receiving light altered by the biological sample and processing the received light to obtain spectrographic data of the biological sample.

In one aspect, the invention provides a method for obtaining a biological sample from a subject. The method may providing a vessel of water, providing a tube into the water and receiving an exhalation of the subject through the tube into the water.

The above description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description or as illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1. is a block diagram illustrating the system architecture;

FIG. 2. is a flow chart to establish model feasibility, development, and use;

FIG. 3. is an upper perspective view of an example probe-sensor unit;

FIG. 4. is a flow chart demonstrating development and use of a Data/Spectral Engine;

FIG. 5. is a flow chart describing the development and use of a Universal Pathogen Family Classification Model Algorithm;

FIG. 6. is a flow chart describing the development and use of a Pathogen Variant Membership Algorithm;

FIG. 7 is a plot of sample data for three covid-19 variants against known negative samples;

FIG. 8 is a plot of sample data for a fourth covid-19 variant against the known negative samples;

FIG. 9. is a flow chart describing how the sample pathogens of interest are determined.

FIG. 10 shows a system for virus detection;

FIG. 11 shows an embodiment of a sensor box;

FIG. 12 shows an embodiment of a housing for a sensor box;

FIG. 13 shows a cuvette and cover that can be used with the sensor box;

FIG. 14 shows an embodiment of the internal components of the sensor box;

FIG. 15 shows the internal components of the sensor box with a cover removed from a sample holder;

FIG. 16 shows a power supply for the sensor box;

FIG. 17 shows a fan port of the sensor box;

FIG. 18 shows a data port of the sensor box;

FIG. 19 shows a first step of loading the sensor box;

FIG. 20 shows a further step of loading the sensor box;

FIG. 21 shows a final step of loading the sensor box;

FIG. 22 shows a sample holder in isolation and a sample holder holding a cuvette;

FIG. 23 shows a system and process for analyzing a sample;

FIG. 24 shows an operational flow for a user to execute an analysis application on a computer;

FIG. 25 shows sample data for a SARS-COV-2 test;

FIG. 26 shows sample data for a HCoV-NL63 test; and

FIG. 27 shows a sampling system.

### DESCRIPTION OF CERTAIN EMBODIMENTS OF THE PRESENT INVENTION

In the following detailed description of the preferred embodiments, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

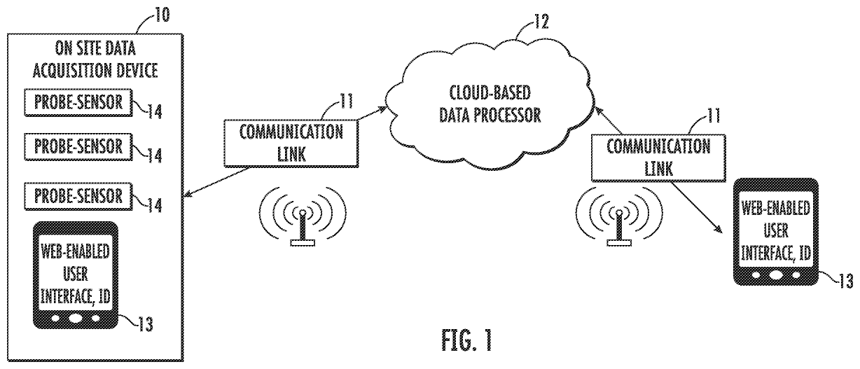

It has been found by the present inventors that spectrographic systems can be used to detect the presence of biological pathogens such as viruses, and particularly corona viruses, in biological samples obtained from subjects. Pathogens can have characteristic wavelength signatures that enable the virus to be detected in biological samples by spectrographic techniques. An embodiment of the invention is described with reference to the figures using reference designations as shown in the figures. FIG. 1 shows the system architecture which comprises at least one on site data acquisition device or probe sensor 10 to interrogate the product or substance in question, a cloud-based data processor (data processor) 12 having at least one computer processor to analyze the data and compute results, a web enabled user interface (user interface) 13 to display results on a user computer device in a format that is accessible to non-experts, and in this embodiment two bidirectional communication links 11, one that sends data between the cloud-based data processor 12 and the on site data acquisition device 10 and the other that sends data between the cloud-based data processor 12 and the web-enabled user interface 13.

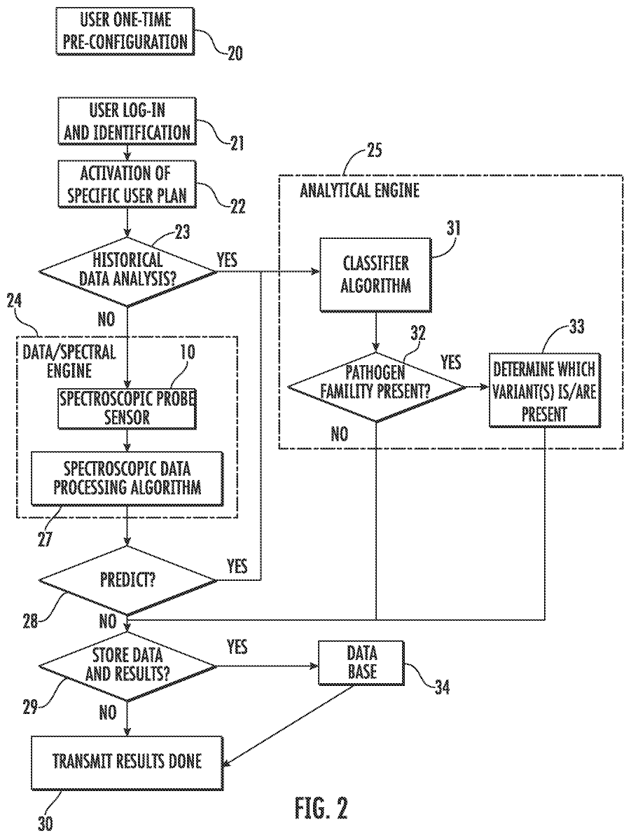

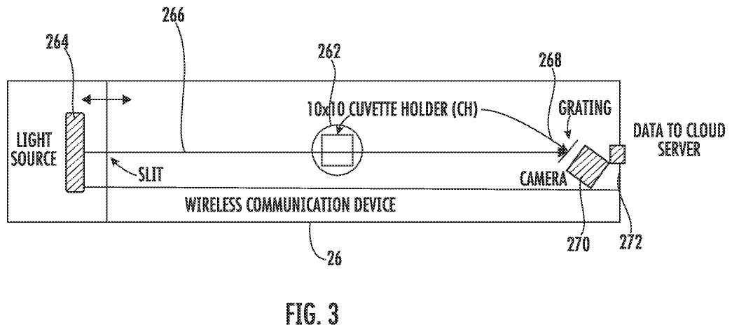

In this embodiment, the data acquisition device 10 is the apparatus further described in FIG. 3 that collects the data from the substance in question and transfers it to the cloud-based data processor 12 via a communication link 11. In the data processor 12, the data is analyzed as further described in FIGS. 2, 4, 5, and 6. The data is then transferred to a web-based user interface which may be installed on any web enabled device including but not limited to a data acquisition device 10 computer, cellular phone, or tablet. The user interface 13 displays the information in a format that makes the results easy to understand for non-experts.

Bidirectional communication links 11 are responsible for connecting the components 10, 12, 13 in FIG. 1 and can include a plurality of telecommunication modes. Buffering capabilities are stored in the user interface, cloud-based data processor, and data acquisition device to ensure signal integrity and retention in the event that the communication network temporarily fails to transmit signals in real-time.

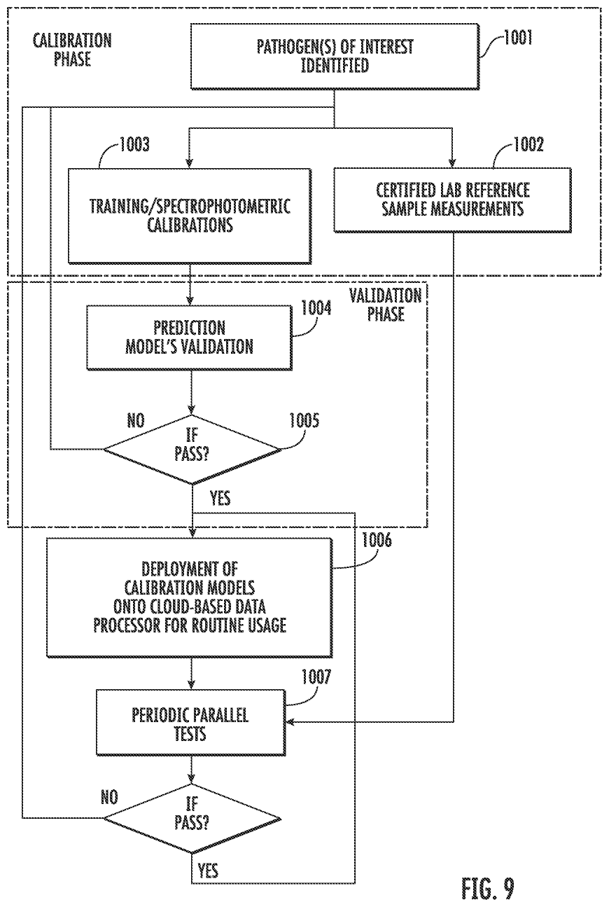

FIG. 9 is a flowchart which demonstrates how the initial parameters of the virus in question are analyzed and applied to the calibration models stored on the cloud-based data processor. For example, the user may wish to configure the system to examine a number of different biological pathogens, such as viruses, each pathogen having its own characteristic wavelength signature or spectrograph. Once the user pre-selects the pathogen of interest 1001, the samples are analyzed in two ways. One is by using a certified lab reference 1002, the other by probing samples from the certified lab in the form of a training set using spectroscopic means 1003. A correlation between the training data set and the certified lab results is identified. A mathematical relationship between the results from the certified lab and the identified spectral points is then developed for the training set. From here, calibration prediction models are used to predict declared pathogens from samples other than the training set 1004. If the prediction errors are beyond an acceptable limit (determination 1005), the prediction model is deemed unacceptable and the process begins again from the training/spectrophotometric calibrations and certified lab reference sample measurements. If the prediction model is deemed acceptable, the model is deployed onto the cloud-based data processor 1006. Tests analyzing specified parameters of interest between a certified lab and the prediction model used in the cloud-based data processor are parallel tested periodically 1007 to ensure there are no drifts. In cases where a periodic test fails, the associated prediction model is sent back to the Training/Spectrophotometric Calibrations step of the process.

FIG. 2 is a flowchart which demonstrates the feasibility of the invention. The process shows the way the data is obtained, computed, stored, and displayed, as well as how the user interacts with the system. During the initial use as shown in block 20, the system undergoes a one-time pre-configuration completed by the user using the web-enabled user interface 11.

During all subsequent uses, the system is initiated through block 21, a user log-in and identification. One object of block 21 is to retrieve historical data and present analytical options specific to the user. Thus the successful application of block 21 automates the activation of block 22 which retrieves the specific user plan. Then, in block 23, the user is given an option to either select data previously collected, or to collect new data to be analyzed.

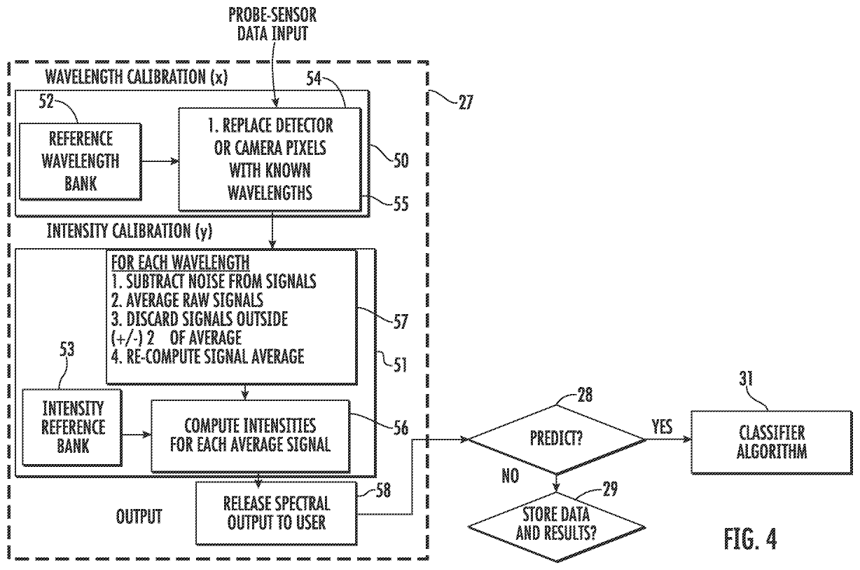

In cases when historical data analysis is not selected, the spectroscopic probe sensor 10 is employed to collect data from the substance in question, such as a biological sample, and transfers that data using a communication link 11 to the cloud-based data processor 12 where the data is analyzed using a spectroscopic data processing algorithm 27 as shown in the data/spectral engine 24 and further described in FIG. 4. Once analyzed in Block 27, control goes back to block 28. Depending on the user preferences selected at the time of system configuration, results are either displayed on the user interface 13 or the data is sent to the classifier algorithm 31 in the analytical engine 25 to undergo further analysis.

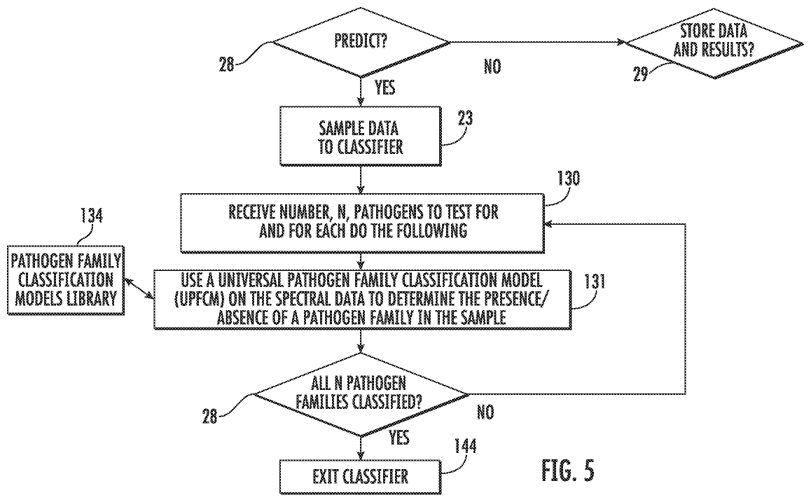

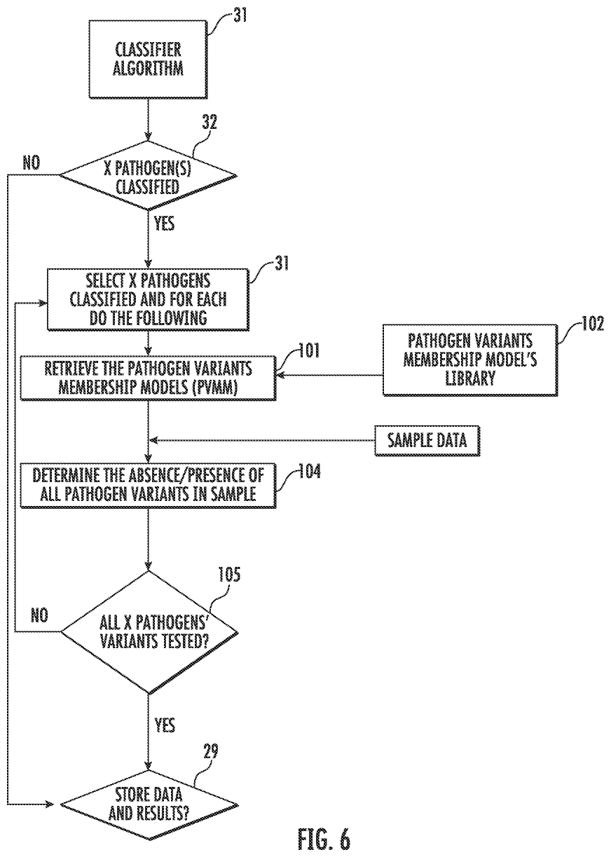

The analytical engine 25 includes two algorithms: the pathogen family classifier algorithm 31 and the pathogen family variants detection algorithm 33. The object of the classifier algorithm 31 is to determine which pathogen families are in the biological sample of interest. After data passes through block 31, it transfers to the pathogen family variants algorithm 33 to determine the presence/absence of specific pathogen family variants. The pathogen family classifier algorithm 31 is further detailed in FIG. 5. In cases where the sample data transfers to the pathogen family variants algorithm 33, it undergoes further interrogation to precisely determine which variants are present in the biological sample. The pathogen family variants algorithm is further detailed in FIG. 6. Once the data in block 33 has been analyzed, it transfers to block 29 where the results may be stored in a database prior to displaying on the web-enabled user interface 13. Alternatively, results may directly transmit to the user interface 13 or any other web-enabled device of choice. Once results are transmitted in block 30, the process has finished.

FIG. 3 is a block diagram of a basic probe sensor data acquisition device 10, which may be used to collect sample data from a product or substance in question. This device operates by shooting light 266 from a light source 264 onto the substance in question. The light beam is then incident upon a grating 268 and finally a camera 270, thereby collecting pixels and signals to obtain a spectrographic sample. The light source 264 may produce light at any wavelength or set of wavelengths targeted at the particular substance or sample in question. For example, the light source may produce light in the visible, UV, near infra-red or infra-red regions. The light source 264 may include multiple sources that produce light in different wavelength bands.

A data processor and communications module 272 may provide some initial processing of the sample data and then communicate the spectrographic data to other components for additional processing. In one embodiment, the sample may be a vial or similar receptacle that is able to receive a biological sample of a subject. For example, the subject may spit or otherwise provide saliva or a nasal swab into the vial. Other biological samples such as blood, urine, sweat, mucus, earwax, etc. may be provided. The vial is placed in the cuvette holder 262 of the apparatus which supports the vial so that the vial interrupts the light beam. Other types of samples and sample collection devices will be apparent to the person skilled in the art. The probe sensor used in this analytical system transfers the data collected to a web-based server, via the communications module 272, through any type of wireless connection device, including but not limited to Wi-Fi, Bluetooth, and cellular radio.

In FIG. 4, the sample data transmitted from the data acquisition device 10 is transferred into the data/spectral engine 27 where the signals received are converted to a suitable data format. The data/spectral engine 27 is located in the data processor instead of the probe sensor. In block 50, a wavelength (x-axis) is assigned based on the average of the pixel readings and is calibrated using a reference wavelength bank 52. Then in block 51, the appropriate intensities for the sample reading are assigned. To initiate this process in block 57, the system subtracts noise from each wavelength signal. The raw signals are then averaged. After this step, any signals that fall within +/−two standard deviations outside of the mean are discarded and the remaining signals are then averaged again. At this stage, the final average for each signal is computed using the intensity reference bank 53 to assign the accurate intensity for the signal. The data is then released to the user through block 58. In block 28, the data either transfers to the classifier algorithm 31 to undergo further analysis, or it displays the results with the option to store them in block 29, depending on the user specifications.

FIG. 5 demonstrates how samples that enter the universal pathogen family classifier algorithm 31 are computed and analyzed. The classifier algorithm is responsible for determining which pathogen families are in the biological sample of interest. In block 23, the sample data are transferred into the classifier. If the pathogen of interest in a biological sample is, for example the corona virus SARS-CoV-2 the data proceeds to block 131 where it is interrogated by a universal pathogen family classification algorithm/model to determine if its present/absent in the sample.

In block 131, the sample data is retrieved along with the parameter membership models library 134. Using the Pathogen Family Membership Models' Library 134, the algorithm searches for pre-identified pathogen variants and if at least one of them is present the outcome would be a POSITIVE for that pathogen family.

After the Universal Pathogen Family classifier has determined the pathogen families in a biological sample of interest for all pathogen families 143, the system initiates the Pathogen Family Variant Memberships Models (PFVMM) algorithm 33, shown in FIG. 6. The objective of the PFVMM 33 is to determine which pathogen family variants are present in the sample of interest, at least one variant must be present if the Universal Pathogen Family classifier output is positive.

In cases where the user programs the system to display results after the classifier, block 32 will automatically send the data to block 29 where results are either stored and displayed or immediately displayed depending on the user's pre-programmed preferences. In cases where the user chooses to run the sample through the PFVMM, block 32 automates the initiation of block 100 where the system identifies all pathogen families that were analyzed in the classifier 31 and automates the PFVMM 33 to determine the pathogen family variants. Like the universal pathogen classifier in FIG. 5, the PFVMM deals with each pathogen variant sequentially. In block 101, the PFVMM retrieves the appropriate equations for the pathogen variants by referencing the Pathogen Variants Membership Models Library 102. The system then receives the sample data 103, and determines the absence/presence of all pathogen variants in the sample in block 104. The system checks the sample has been analyzed for all variants in block 105. If not, the system initiates again in block 100 to select a remaining variant, and will run through this cycle (block 100-105) until all variants have been checked. Once analyzed, the data results proceed to block 29 where the system computes whether to store the data or immediately display results depending on the user preference.

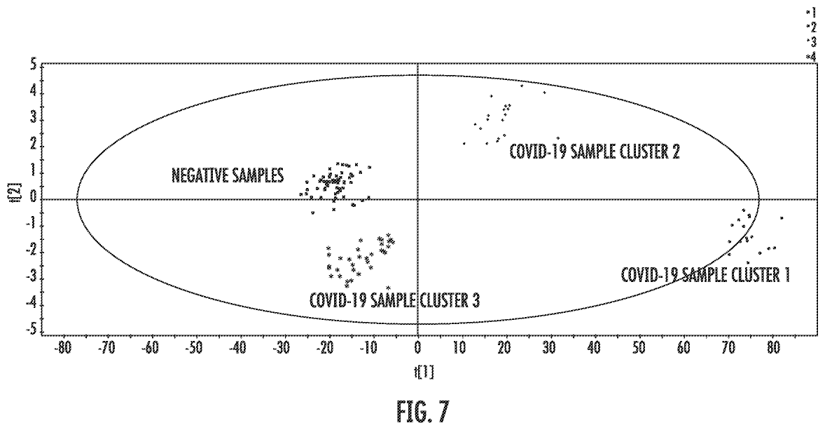

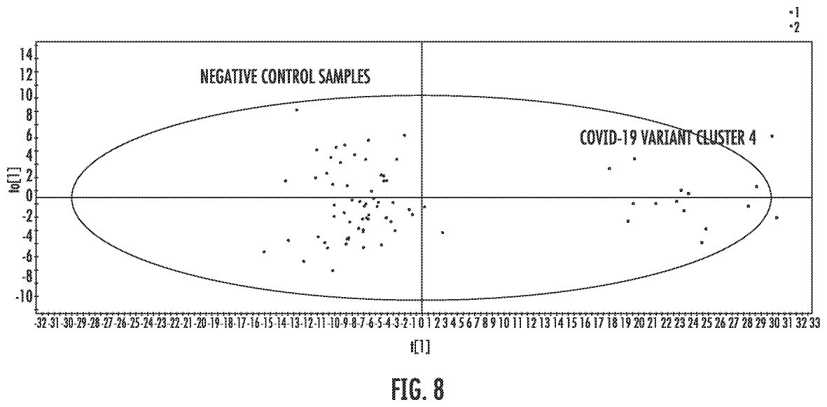

FIG. 7 shows a 2-D Plot of three Covid-19 Variants clusters and Negative Control Cluster produced from a number of samples using the methods described herein. A fourth Covid-19 variant produced results too close to the negative samples to be displayed in FIG. 7. However, this variant can be shown clearly distinguished in the plot of FIG. 8. FIGS. 7 and 8 show not only that the system is able to clearly distinguish between positive covid-19 samples and negative samples (known controlled negative), but the very delineated clusters demonstrates that the system is also able to clearly distinguish between different variants of the covid-19 virus.

As the PFVMM algorithms identify variants within each pathogen family the results are better compared to “bundling” all the variants in one algorithm. In a blind test against the stand RT-PCR test, the present methodology was shown to be 94% if exclude borderline positive samples were excluded as positives, and 100% accurate the borderline positives samples were included as positives.

Class discriminant equations are developed for both the universal pathogen classifier, and the pathogen family memberships such that they assign numerical values expressing the probability of membership in the classes being tested for or neither. The discriminate equations use pre-selected wavelengths' output intensities, λ₁, λ₂ etc., and pre-assigned coefficients associated with each of the selected wavelengths, a₁, a₂ etc.
Let c₀ be a pre-determined constant associated with a class discriminant equation
Member Score=c₀+a₁λ₁+a₂λ₂+ . . . .
When a sample is spectroscopically scanned the system will retrieve the intensities associated with wavelengths λ₁, e.g. 550 nm, and λ₂, e.g. 622 nm, and input, and evaluate either the presence/absence of a pathogen family (UPFMM), or its variant (PFMM)
For example:

| | | Borderline | |
|---|---|---|---|
| | Member If | Member If | Not a Member |
| Member Score | .50 <= | .35 < MS < .50. | MS > |
| (MS=) | MS <= 1.35 | | 1.35, MS < .35 |

A user may pre-register for an account with an online analysis lab. The user may enter into a payment plan with the online analysis lab. For example, the user may pay a fixed amount per month or may pay on a per-use or other basis. The user's payment may entitle the user to a number of analysis services, a period of analysis services, or a combination. Specific registration and payment plans are not considered pertinent to the present invention and with online registration systems being well established for many internet-based services, no further description of the registration process is considered necessary herein.

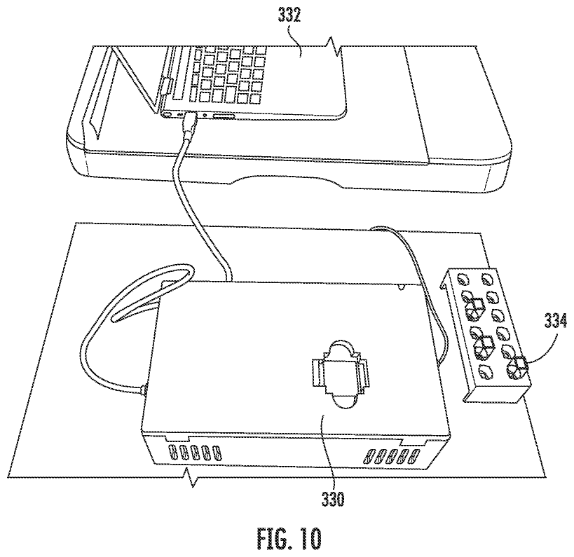

FIG. 10 shows actual sample apparatus for an embodiment of a virus test system in accordance with the present disclosure. The system includes a BMx Model A Analyzer/Sensor Box 330, a computer with access to the internet 332 and disposable cuvettes 334. The computer system may be programmed with a BMx Cloud Real-Time COVID Test Subscription that enables sample data to be uploaded and processed in real-time to generate a result on the sample.

The Sensor Box and the computer do not have to be in proximity as shown in the figure. They can be as far part as possible, or even in different rooms/cubicles, depending on the length of the USB cable used. In addition, wireless connectivity between the sensor and the computer may be implemented.

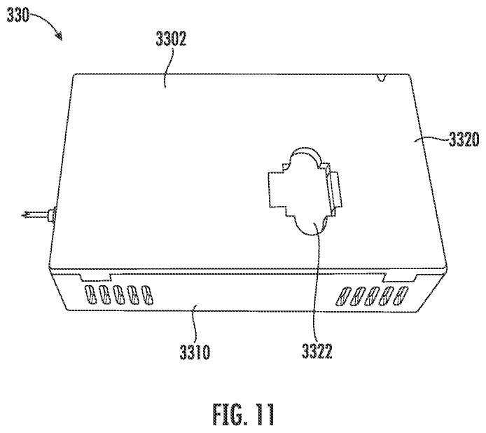

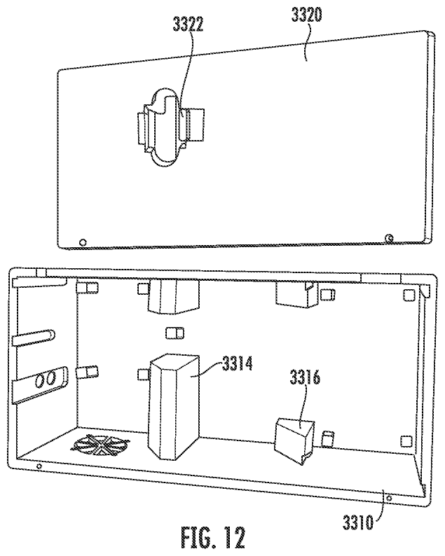

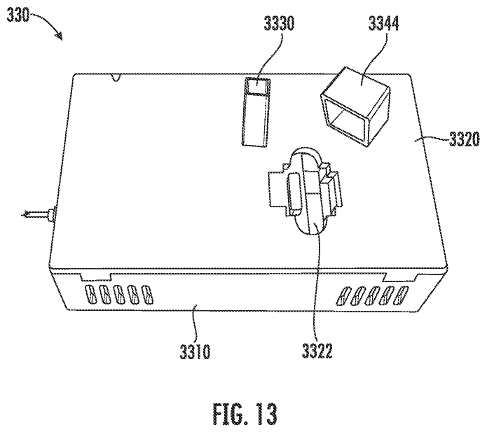

FIG. 11 shows an embodiment of the sensor box 330 for receiving and analyzing samples. The sensor box 330 includes a housing 3302. The housing 3302 includes a chassis 3310 and a lid 3320 (FIG. 12). The chassis 3310 is considered a drop and stick chassis with internal formations (e.g. projections, slots, recesses, and divisions, such as projections 3314, 3316) that are configured to receive and hold the components of the sensor box. The chassis lid 3320 may be secured to the chassis 3310 by any suitable means including a hinge joint (as shown), screws, latches, press fit detents, etc. or a combination of such methods. The chassis lid 3320 includes a sliding cover 3322 that can slide open to provide access to a sample chamber or slide closed to cover the sample chamber and prevent ambient light from entering the sample chamber. When open, as shown in FIG. 13, a cuvette 3330 containing a sample may be inserted into a sample holder (described below) within the sensor box 330. A cuvette cover 3344 may be placed on the sample holder to encase the cuvette containing the sample. The chassis 3310 and chassis lid 3320 may be 3-D printed, plastic molded or formed by any other suitable processes.

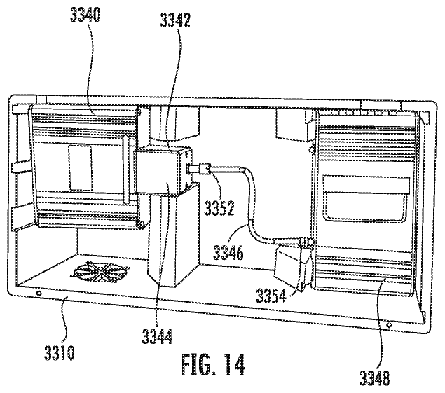

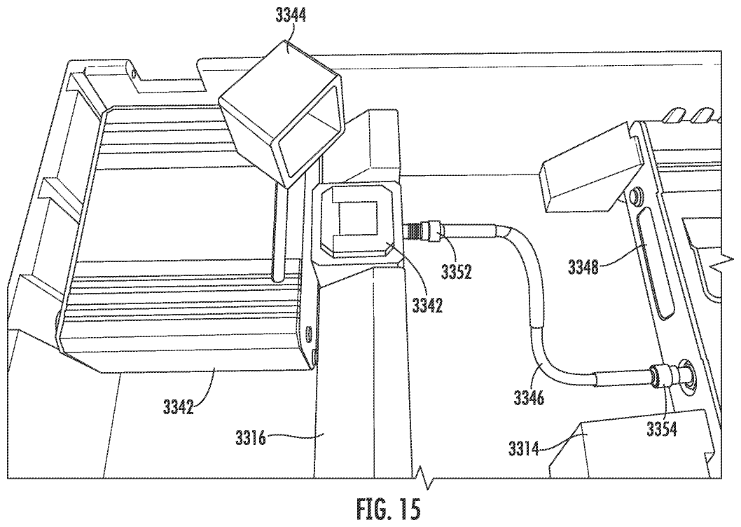

As stated above, the chassis 3310 includes internal formations (e.g. projections, slots, recesses and divisions) that are able to receive and secure the components of the sensor box 330. FIG. 14 shows an internal view of the chassis 3310 including the working components of the sensor box 330. The sensor box 330 includes an AC powered lamp 3340, a sample holder 3342 that receives the cuvette 3330 containing a sample, cuvette cover 3344, optical fiber 3346 and spectrometer 3348. The internal formations within the chassis 3310 allow for precise fit of these components thereby removing or at least minimizing the need for additional fastenings, such as screws. However, additional fasteners can be utilized if preferred. FIG. 14 shows the sample holder 3342 with the cuvette cover 3344 in place. FIG. 15 shows the sample holder 3342 with the cuvette cover 3344 removed to enable the sample holder 3342 to receive a cuvette 3330.

The sample holder 3342 includes an aperture (not shown) in one side of the sample holder 3342. The lamp 3340 abuts the sample holder 3342 so that light is projected directly from the lamp 3340 into the sample holder 3342 within minimal stray light from the lamp 3340 escaping into the chassis cavity. Thus, light from the lamp can illuminate a sample within a cuvette located in the sample holder. An inlet end 3352 of the optical fiber 3346 connects to an opposite side of the sample holder 3342 from the lamp 3340. An outlet end 3354 of the optical fiber 3346 connects to the spectrometer 3348. Thus, the optical fiber 3346 is able to conduct light that has passed through a sample within the sample holder 3342, and is thus encoded with the sample wavelength signature, from the sample holder to the spectrometer 3348 for analysis by the spectrometer 3348.

As stated above, the lamp 3340 may emit light across a range of wavelengths in in the Visible and near infrared. In one embodiment, the lamp 3340 may be a halogen lamp that emits light in the wavelengths 400 to 1100 nm. In one embodiment, the spectrometer may be a BLUE-Wave™spectrometer available from StellarNet Inc.

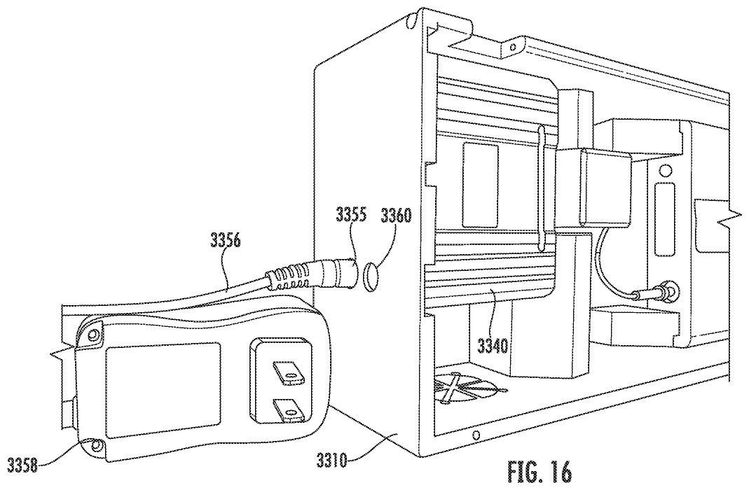

The lamp 3340 includes an inlet power port and a switch on one side of the lamp. As shown in FIG. 16, the chassis 3310 may be provided with an aperture 3355 that allows a connection of a power cable 3356 for a power supply 3358. A further aperture 3360 may provide access to the lamp switch.

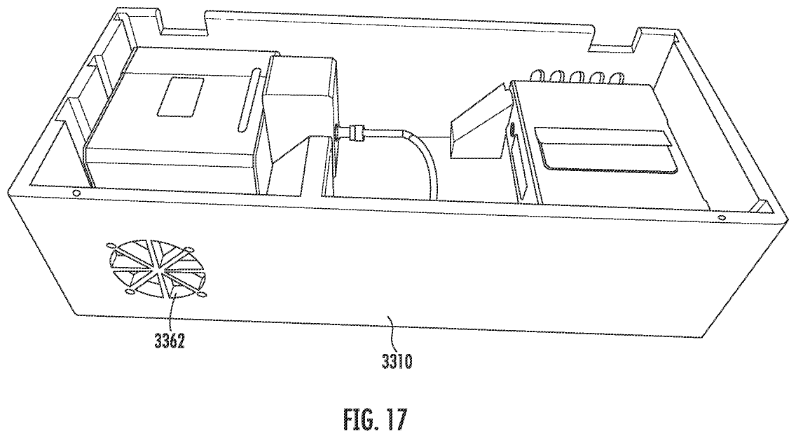

On a side of the chassis 3310, there may be provided a fan port 3362 (FIG. 17). A fan (not shown) may optionally be provided adjacent to the lamp 3340 for cooling the lamp and other components.

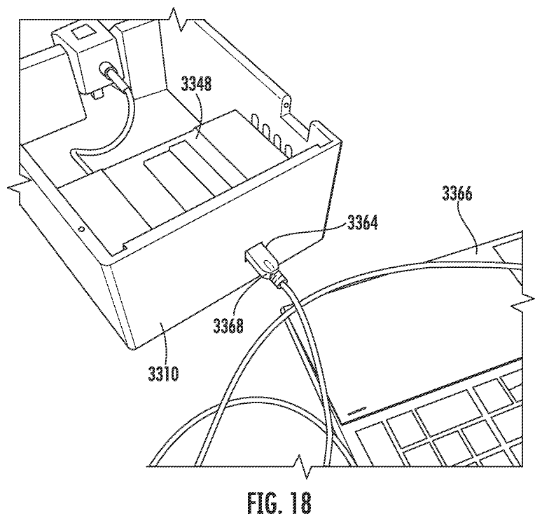

In the chassis 3310 adjacent the location of the spectrometer 3348, a data port 3364, such as a USB port, allows a connection of the spectrometer 3348 to an external computer 3366 via a cable 3368 (FIG. 18). A computer may be used to extract data from the spectrometer and to execute analysis programs. A USB connection can also be used to provide power to the spectrometer.

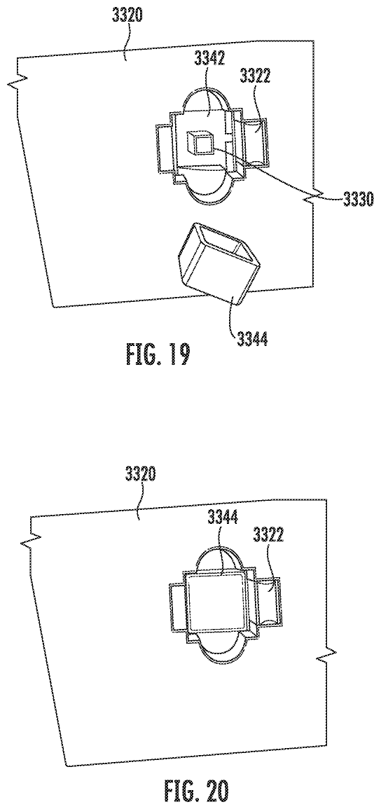

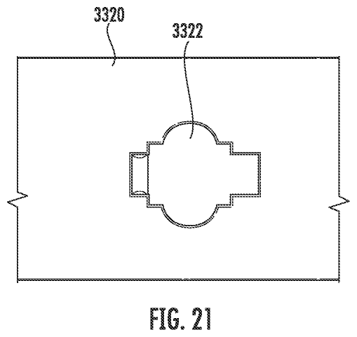

The sensor box may be operated by first placing a liquid sample in the cuvette 3330 and then locating the cuvette into the sample holder 3342 (FIG. 19). The cuvette cover 3344 may then be placed over the cuvette to seal the sample holder 3342 from ambient light within the chassis (FIG. 20). The sliding lid 3322 may then be closed to seal the sensor box 330 from external light (FIG. 21).

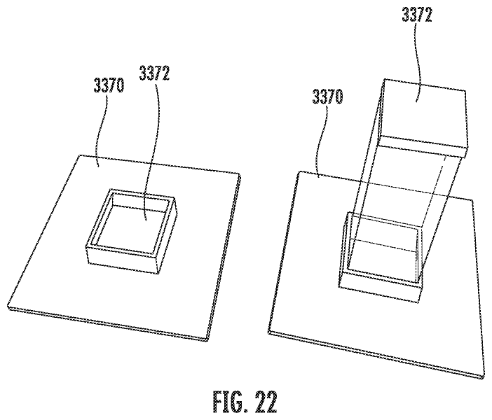

FIG. 22 shows the sample holder 3342 in more detail, including its engagement with the cuvette 3330. The sample holder 3342 includes a base 3370 with a square aperture 3372 that receives a square-based cuvette 3330. The cover 3344 is located over the base and encases the cuvette. Typical cuvettes are round bottomed and fall over easily, or must be supported in a cuvette rack to prevent spectrometer sample spills on work benches. By contrast, the cuvette 3330 is square bottomed and designed specifically for interaction with the sample holder. The cuvette may allow a user to spit saliva for safe and isolated testing, in particular for covid sampling.

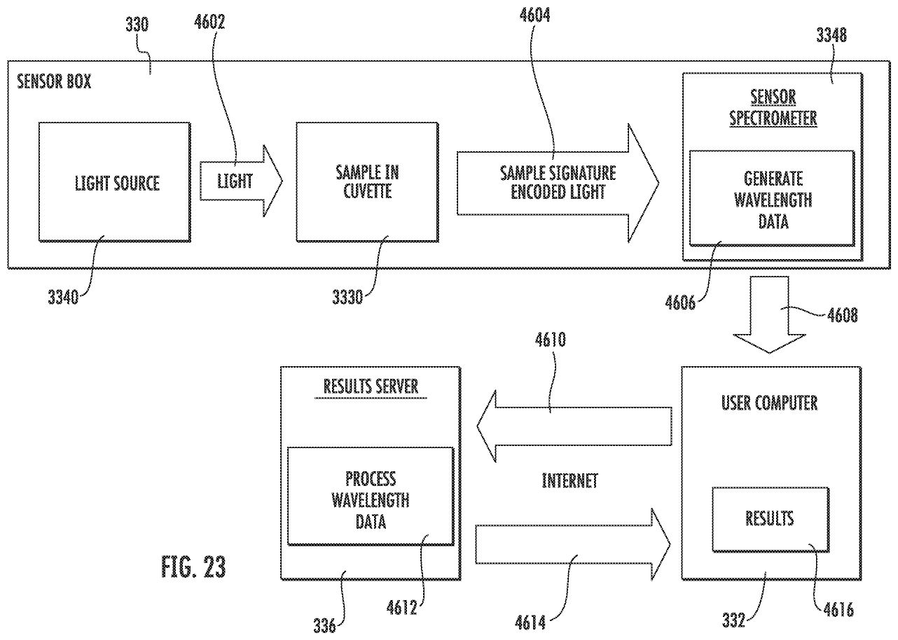

FIG. 23 shows a process for spectroscopic analysis of a sample. The Sensor Box collects optical data from the whole organism/virus structure leaving it intact. The computational components process the optical data by searching for optical resonant frequencies or wavelengths in the sample data consistent with SARS-COV-2 molecular signature, or the particular virus under examination, to determine if it is absent/present in the sample. Using all data possible the analyzer can differentiate a virus even from its genetic family “relatives” thus minimizing false positives/negatives. With specific reference to FIG. 23, at step 4602, light from light source, e.g. lamp 3340, is projected through a sample in the cuvette 3300. The light source produces light in a spectrum of wavelengths sufficient to produce the wavelength signature for the particular pathogen under investigation. Wavelengths may include, without limitation, visible light, UV, infra-red, etc. At step 4604, light altered by the sample, i.e. encoded by the sample signature, is received into the spectrometer 3348. The spectrometer 3348 processes the sample light signature into wavelength components (step 4606) which are passed to a computer 332 (step 4608). The computer 332 passes the spectrum data to a results server 336 over a suitable communications channel such as the internet (step 4610). The server 336 processes the spectrum (step 4612) by applying algorithms pertaining to the pathogen under investigation to determine if the sample spectrum indicates the presence of the pathogen under investigation. The results are passed from the server 348 to the computer 346 (step 4614) for display to the user on the user interface (step 4616). Typically, the results display will be a relatively simple indication that the sample is positive/negative to the pathogen of interest. Additional data, including quantitative data, confidence levels, etc. may also be displayed.

The processing of the sample data has two components to it, an Offline and Online.

Offline:

Multivariate Data Analysis is specifically used, or is one method example used, to identify the virus of interests resonant/discerning wavelengths that would differentiate it from other viruses. In one embodiment, SARS-CoV-2 was the target virus and a goal was to find wavelengths to differentiate it from another coronavirus i.e. HCoV-NL63, also its genetic cousin.

Online:

(ii) Multiple Linear Regression (MLR) methods are used to determine if the virus of interest is positive in a test sample using wavelengths, determined offline, as independent variables (x). MLR output y is given as

y=β₀+β₁x₁+β₂x₂+ . . . +βₙxₙ

Where b₀ is the regression constant;
b₁ to bₙ are regression coefficients, and
x₁ to xₙ are the virus resonant/discerning wavelengths.

The MLR output (y) is used as an indicator if the virus is present/absent. The MLR models diagnose the samples and if the output, y, outputs positive value then the SARS-CoV-2 is present. Positive output is a range e.g. between 0.5 and 1.35.

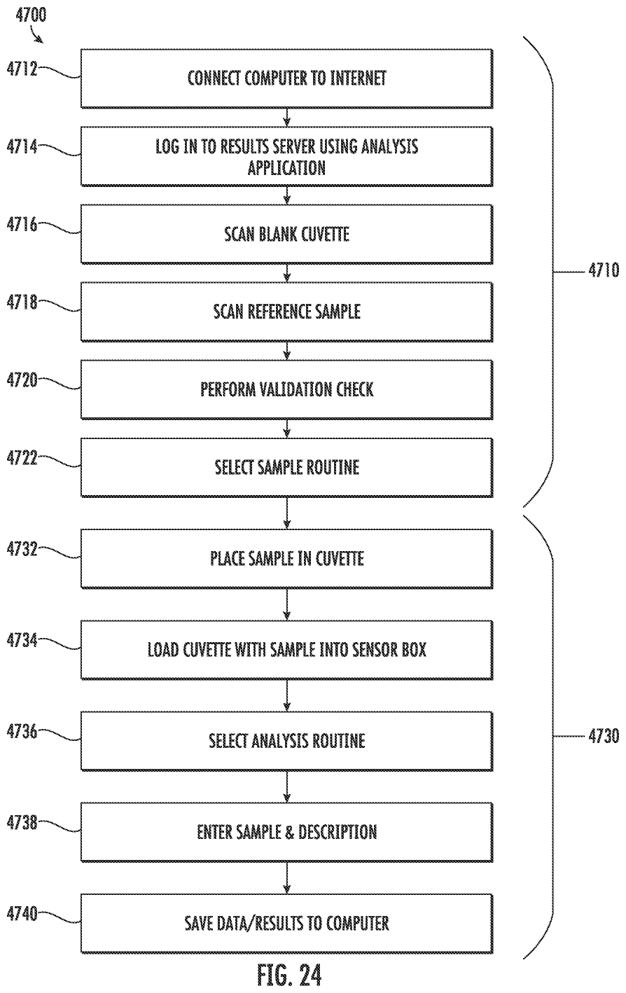

FIG. 24 shows the analysis process coupled with the test apparatus. The process 4700 includes an initialization phase, and a sample test phase. Unlike prior art pathogen test systems, in particular for coronavirus analysis, the test phase can be expected to take approximately 5 minutes to produce a meaningful indication of the presence of a pathogen under investigation within a sample. At step 4710, the analyzer may be initialized. Initialization may include connecting the user computer 332 to the internet (step 4712) and logging on to the results server 336 via a specific sample application or user interface on the user computer 332 (step 4714). The analyzer may be calibrated by scanning a blank cuvette with the light off 4716, scanning a reference fluid (e.g. distilled water) in a cuvette (light on) 4718 and performing a validation check 4720. The user may then select a sample routine on a GUI of the user computer 346 (step 4722) including selecting the pathogen or virus to be tested for. Selection of the pathogen in turn selects the wavelengths and coefficients used for the MLR model. The analysis application executing on the user computer 332 may provide a series of prompts to guide the user through the initialization phase.

In the sample analysis phase 4730, a cuvette is filled with sample saliva, or other body fluid that can provide an indication of the pathogen under test (step 4732). The cuvette is loaded into the sensor box 330 (4734). The GUI on the user computer 346 may direct these steps. At step 4736, the user selects to scan the sample on the GUI, which causes the user computer to actuate the spectrometer within the sensor box. As soon as the user clicks on the Graphical User Interface, the process continues automatically between the sensor box, computer and results server, with the spectrometer providing spectral data to the computer which is then forwarded to the results server, and with the computer receiving results data from the results server. At this time, the user may enter a Sample ID and description for the sample, such as details of the patient, time, location of test, etc. (step 4738). The user may then store the received results against the sample ID 4740. The results are received relatively quickly, as fast as within a few seconds. They are output as either positive/negative/inconclusive with no need for the user to read and interpret complex scientific codes.

An advantage of this system is that the tests require no sample preparation—patients can even load their own untreated saliva samples into the sensor box test port. Thus no highly skilled personnel are needed, and results are output after just a mouse click on the GUI of the analysis application.

The test system described is able to detect and distinguish between various viruses at a time. Experiments by the present applications have already demonstrated the ability to distinguish between SARS-CoV-2 (Covid-19), and HCoV-NL63 (Common Cold) viruses and healthy saliva simultaneously. This is particularly critical since COVID-19 and the common cold exhibit similar symptoms. Preliminary work indicates that the limits of detection for accurate results could be as low as 1000 infectious virus particles per milliliter (1000 ivp/mL).

In addition to the SARS-COV-2, the same spectral data for a sample can be used to test for the Human coronavirus NL63, thus removing the need for multiple sampling.

Other viruses akin to SARS-COV-2 can easily be misdiagnosed as SARS-COV-2. These include:
The Flus virus
Human coronavirus 229E
Human coronavirus OC43
Human coronavirus HKU1
SARS-coronavirus
MERS-coronavirus

It is advantageous that the presently described systems and methods can be used to simultaneously test these additional pathogens using just one sample. Moreover, there is no limit as to how many viruses the analyzer can test for.

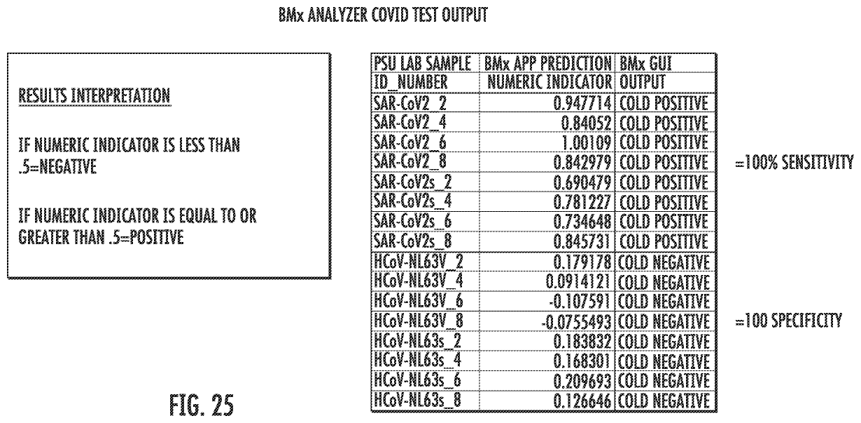

FIG. 25 shows the Analyzer output for a Covid test using the MLR algorithm specific to the SARS-CoV-2 virus, i.e. with specific wavelengths and coefficients. Each sample analyzed is assigned a numeric score/indicator if it is positive or negative. In the example below a numeric indicator or score between 0.5 and 1.35 means that the sample is positive, and in this case specifically SAR-CoV-2 is present.

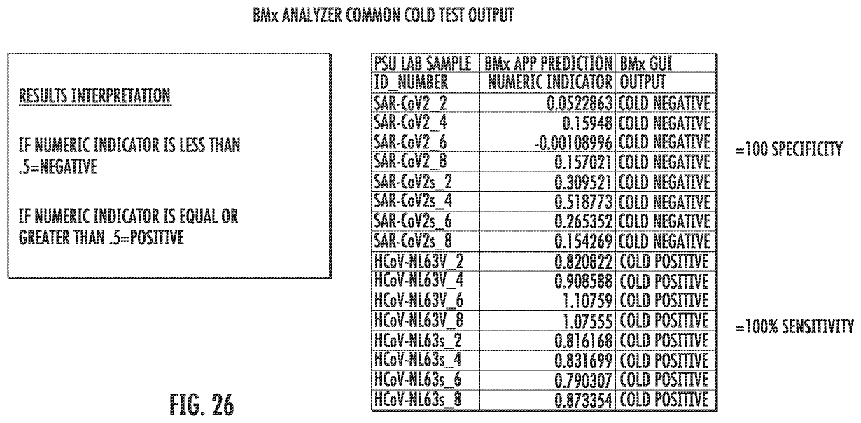

FIG. 26 shows the Analyzer output for Common Cold test (i.e. with a different set of wavelengths and coefficients than for the covid test of FIG. 25. As for the covid test, each sample analyzed is assigned a numeric score/indicator if it is positive or negative. In the example below a numeric indicator or score between 0.5 and 1.35 means that the sample is positive, and in this case specifically HCoV-NL63 is present. FIGS. 25 and 26 show how collectively, different MLR models can be applied to a sample to determine different viruses within the sample. The present examples demonstrate how SARS-COV-2 virus, and other viruses can be spectrally isolated/resolved so that subjects not infected by the target virus will not be mistaken as having the targeted disease, e.g. COVID-19. The analyzer system is able to identify and sort the specific wavelengths required to isolate the SARS-COV-2 virus from its genetic cousins and other viruses.

The spectroscopic analysis systems and methods described have many advantages over more complicated testing systems, in particular PCR test methods. Advantages include:

REAGENTS AND CONSUMABLES: No reagents needed/no supply chain issues, just distilled water needed.

NON-DESTRUCTIVE 100% DIRECT TESTS: The PCR test method destroys the virus with chemicals while the present optical technology samples the whole virus intact, without interfering/sample degrading chemicals, to extract maximal biochemical/biomolecular information.

THROUGHPUT: Each sensor box can produce 100 real time results per 8-hour shift.

COST: Current tests (PCR, Antigen etc.) can cost as high as $150 per test while the cost per sample test for the present optical technology can be as little as $5 for the same Covid test.

PERSONNEL: Highly skilled lab technicians are required to administer current tests such as PCR and Antigen Tests. By contrast, anyone can operate the sensor box analyzer including clericals, admins, patients etc. . . . .

FOOTPRINT: The sensor box is about the size of a shoebox and weighs approximately 3 pounds thus can easily be taken to the patients or from place to place

WORKER SAFETY: The Sensor Box and the computer can be as far part as possible, or even in different rooms/cubicles, depending on the length of the USB cable used. The patients can even load their own saliva samples.

REAL-TIME RESULTS RELEASE: The sensor box analyzer results are produced almost instantly, while PCR and Antigen Tests results can take from hours to days.

RESULTS INTERPRETATION: Expressed plain English i.e. Positive/Negative.

The sensor box system and computer application can be distributed to multiple sites for ready implementation of a high intensity testing regime. Locations may include rapid transit points such as airports, bus terminals, etc. . . . . The real-time analyzer output makes it possible to avoid detaining passengers while waiting for results. Public organization such as schools, universities, and colleges, which may have tens of thousands of students may not be able to sustain test costs at $150 per individual/sample on a weekly basis, compared to the lower $5 charge per sample charge. People with modest income will find the analyzer tests much more affordable/accessible and further, people troubled by invasive and painful throat and nose swabs may find the spectroscopic saliva tests more acceptable, where they only spit for the saliva sample to be collected.

The present methods require the collection of a sample from a subject.

Many collection methods for pathogens are indirect e.g. via mouth, throat or nasal swabs etc., and saliva etc. For pathogens that invade the lungs, such as coronaviruses, the pathogen of interest may not be in any of these sampling locations/media but may still be present in the lungs. Indirect oral and nasal sampling of lung pathogens could lead to false negatives while directly sampling exhaled materials from the lungs could improve tests results. An alternative sample collection method will thus be described herein. The lung sample extraction method is intended to minimize false negatives and ensure that any air transportable samples of interest, which include pathogens, viruses and coronaviruses such as SARS-COV-2, are directly extracted from the lungs for testing.

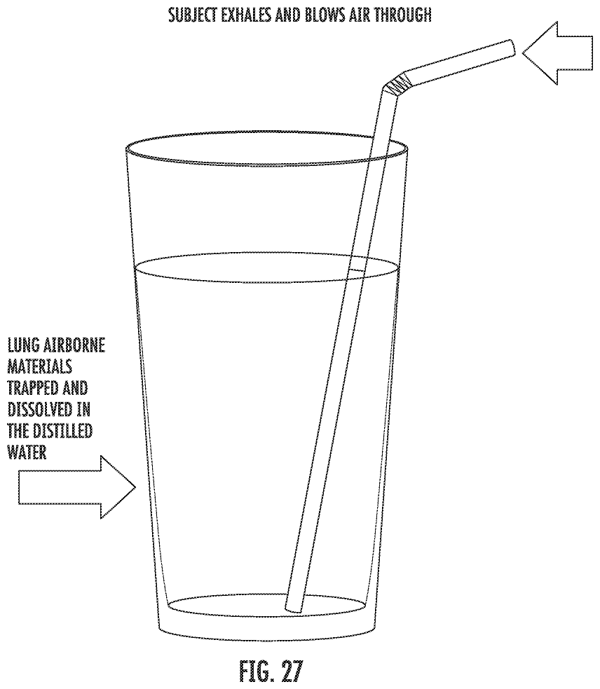

FIG. 27 shows an embodiment of a sample collection method. In the method, distilled water 2702 is placed in a container 2704, such as a beaker, cuvette, vial etc. A tube 2706, such as a straw is placed into the water 2702 and the subject exhales and blows lung air through the tube/straw 2706 where the exhaled gases are dissolved, and particulate material becomes trapped in the distilled water. The “impregnated” distilled water container 2704 containing trapped lung materials can be placed directly into the spectrometer or transferred to a more suitable container, such as a cuvette. Samples collected in this manner may be used for applicable lung, and lung-related tests as samples in a spectrometer or other amenable sampling systems. Examples include tests for pathogens that can reside in the lungs and gaseous indicators/markers of other diseases or other conditions.

Although the description above contains many specifications, these should not be construed as limiting the scope of the invention but as merely providing illustrations of some of the embodiments of this invention. Thus, the scope of the invention should be determined by the appended claims and their legal equivalents rather than by the examples given.

What is claimed is:

1. A method for determining the presence of a biological pathogen in a subject comprising:
(A) obtaining a biological sample from the subject;
(B) obtaining spectrographic data from the biological sample;
(C) determining by a data processor and from the spectrographic data, the presence of one or more biological pathogens in the biological sample.

2. The method of claim 1 wherein determining the presence of one or more biological pathogens in the biological sample comprises:
(A) selecting a biological pathogen of interest;
(B) performing a classification procedure on the spectrographic data that determines if a specified pathogen family is present in the biological sample;
(C) performing a membership procedure on the spectrographic data that determines if at least one pathogen variant is present in a pathogen family.

3. The method of claim 2 wherein performing the classification procedure comprises:
(A) executing a Universal Pathogen Family Classification Model to determine the presence/absence of a pathogen family in a sample.

4. The method of claim 2 comprising performing a pathogen family variant membership test procedure for the identified biological pathogen family that calculates a membership score.

5. The method of claim 4 wherein performing the membership test procedure comprises:
(A) retrieving at least one equation for the determined pathogen family from a library;
(B) executing the at least one equation to calculate the membership score value.

6. The method of claim 4 comprising performing the membership classification procedure for each biological pathogen variant of interest.

7. The method of claim 1 comprising selecting a biological pathogen of interest, wherein the selection determines a Multiple Linear Regression equation to be used for analyzing the biological sample.

8. The method of claim 1 comprising:
(A) receiving the biological sample into a sensor unit comprising a light source and a spectrometer;
(B) obtaining the spectrographic data within the sensor unit;
(C) communicating the spectrographic data from the sensor unit to a results server;
(D) processing the spectrographic data in the results server to determine a result indicating the presence of the one or more biological pathogens in the biological sample; and
(E) communicating the result from the results server to a computer coupled to the sensor unit.

9. The method of claim 1 comprising processing the spectrographic data in the data processor to assign wavelength and wavelength intensities to the spectrographic data.

10. The method of claim 1 wherein the one or more biological pathogens comprise one or more viruses.

11. The method of claim 10 wherein the one or more viruses comprise one or more coronaviruses and their variants.

12. The method of claim 1 wherein obtaining a biological sample from the subject comprises:
(A) providing a vessel of water;
(B) providing a tube into the water;
(C) receiving an exhalation of the subject through the tube into the water.

13. A system for analyzing a biological sample comprising:
(A) spectroscopic apparatus configured to receive a biological sample and dispose the biological sample in a light beam to obtain spectrographic data of the biological sample;
(B) a data processor programmed to:
(a) receive the spectrographic data from the spectroscopic apparatus;
(b) determine the presence of at least one selected biological pathogen in the biological sample from the spectrographic data.

14. The system of claim 13 wherein the data processor is programmed to:
(A) communicate the spectrographic data to a results server;
(B) receive a result from the results server that indicates the presence of at least one selected biological pathogen in the biological sample from the spectrographic data; and
(C) display the result.

15. The system of claim 14 wherein the data processor is programmed to:
(A) execute a user interface that enables a user to select a biological pathogen;
(B) communicate the selection to the results server;
(C) wherein the selection of the biological pathogen determines a Multiple Linear Regression equation to be used for analyzing the biological sample.

16. A sensor unit comprising:
(A) a sample holder for receiving a biological sample;
(B) a light source configured to direct light into the biological sample;
(C) a spectrometer for receiving light altered by the biological sample and processing the received light to obtain spectrographic data of the biological sample.

17. The sensor unit of claim 16 wherein the sample holder comprises a base and a cover, wherein the base is configured to receive and retain a cuvette, wherein the cover is configured to enclose the cuvette to prevent stray light from entering the biological sample.

18. The sensor unit of claim 16 wherein the spectrometer is configured to communicate spectrographic data of the biological sample to a computer.

19. The sensor unit of claim 16 comprising a housing, wherein the housing comprises a slidable cover that in an open position allows a cuvette to be loaded into the sample holder and in a closed position prevents light from entering the chassis.

20. The sensor unit of claim 16 wherein the sample holder is configured to receive an end of an optical fiber for conducting the light altered by the biological sample to the spectrometer.

21. The sensor unit of claim 20 configured to be coupled to a data processor programmed to:
(A) execute a user interface that enables a user to select a biological pathogen;
(B) receive the spectrographic data from the spectroscopic apparatus;
(C) communicate the selection of biological pathogen and the spectrographic data to a results server;
(D) receive a result from the results server that indicates the presence of at least one selected biological pathogen in the biological sample from the spectrographic data; and
(E) display the result;
(F) wherein the selection of the biological pathogen determines a Multiple Linear Regression equation to be used for analyzing the biological sample.
